(19) United States
(12) Patent Application Publication
(10) Pub. No.: US 2022/0061608 A1
(43) Pub. Date: Mar. 3, 2022

(54) Germicidal Floor Cleaner

(71) Applicant: Team International Group of America Inc., Miami Gardens, FL (US)

(72) Inventors: Uri Murad, Hollywood, FL (US); Laurent Bardot, Miramar, FL (US); Maik Pezold, Guangdong (CN)

(73) Assignee: Team International Group of America Inc., Miami Gardens, FL (US)

(21) Appl. No.: 17/458,530

(22) Filed: Aug. 26, 2021

Related U.S. Application Data
(60) Provisional application No. 63/074,002, filed on Sep. 3, 2020.

(51) Int. Cl. A47L 7/00; A47L 9/14; A47L 9/12; A47L 9/04; A47L 9/28; B01D 46/00; B01D 39/10; B01D 46/10; A61L 2/10; A61L 2/26; A61L 9/20; A61L 9/14; A61L 9/22

(52) U.S. Cl. CPC A47L 7/0061; A61L 2209/14; A47L 9/122; A47L 9/149; A47L 9/0472; A47L 9/0477; A47L 9/2884; A47L 9/2873; A47L 9/2894; A47L 9/2852; B01D 46/0023; B01D 46/0028; B01D 46/0038; B01D 39/10; B01D 46/10; A61L 2/10; A61L 2/26; A61L 9/20; A61L 9/14; A61L 9/22; A47L 2201/04; A47L 2201/022; B01D 2239/0442; A61L 2202/11; A61L 2202/121; A47L 9/1409

(57) ABSTRACT

A surface cleaning appliance has a motorized blower adapted to pull air containing the genus and germ-laden dust through an internal dust bin. The dust bin includes a chamber, a UV lamp disposed within the chamber, a copper mesh filter, and a HEPA filter. The appliance has a horizontally-rotating roller brush at its intake opening and has an ion generator and an essential oil diffuser at its exhaust opening. A smartphone app enables remote and varied operation of the appliance.

### FIELD OF THE INVENTION

The invention relates to floor cleaning appliances. More specifically, it relates to floor cleaning appliances with germ-killing capabilities, such as vacuum cleaners and floor sweepers.

### BACKGROUND

Vacuums and other types of floor cleaning appliances are well known. Typically, germs taken from the floor are present in air drawn through such appliances, are held in dust captured by filters of such appliances, and in dust and debris collected in dust bins of such appliances. Such appliances are also known to include means for removing certain of those germs from air returned to the room, such as by HEPA filtration, but in light of the recent pandemic, users have become more concerned with and knowledgeable about germ pollution and concerned with the appliances themselves harboring such germs. And since the germ-removing effectiveness of such appliances is impossible for the user to confirm, trust is a critical factor and the use of additional and varied means for removal and/or killing of a wider range of germ types is more important now than ever. Users have become less trustful of mechanical filtration or even other limited systems for germ removal.

To date, no floor cleaning appliance has been capable of providing various means in a series of steps to remove and/or kill virtually all floor-dwelling germs and germs dwelling within the appliance. This inability is due to such factors as the high airflow rate through such appliances, to the fleeting access such appliances have to that airflow to effect treatment of the germs passing there-through, to the often-ideal germ-promoting environments within such appliances, to the inherent inaccessibility of dust collected in such appliances, and to the fact that such cleaning appliances tend to move too fast over the surfaces that they treat to have any efficient germicidal effect.

There exists the need to provide, and such may be an object of the invention, a floor cleaning appliance having a plurality of varied means for removal and/or killing of a wide range of germ types both in the air flowing there-through and to the environment there-in.

Further needs and objects of the invention will become apparent upon a review of the following disclosure of an exemplary embodiment.

### SUMMARY OF THE INVENTION

The invention may be embodied in or practiced using a floor cleaning appliance having a plurality of varied means for removal and/or killing virtually all floor-dwelling germs drawn into the appliance, through a combination of mechanical filtration and germ-killing electronic technologies.

The invention may be embodied in or practiced using a surface cleaning appliance having a housing including; an intake opening, an exhaust opening, a dust bin in communication with and disposed between the intake and exhaust openings and adapted for retaining airborne germs and germ-laden dust, and a motorized blower adapted to pull air containing the germs and germ-laden dust into the dust bin through the intake opening where the germs and germ-laden dust are removed therefrom and to expel the air from the dust bin through the exhaust opening.

The dust bin may include a UV lamp and first and second filters; and the air containing the germs and germ-laden dust may be pulled into the dust bin and through the intake opening where it is bathed in UV light from the UV lamp, may pass through the first and second filters, and may leave the bin through the exhaust opening. The dust bin may include a chamber adapted so that air is circulated around therein for a prolonged period to increase direct UV exposure thereof.

The UV lamp may also directly bathe interior portions and components of the bin to expose any germs retained therein to the UV light. The UV light may be UV-C.

The first filter may be a germicidal filter. The germicidal filter may be a copper mesh filter. The second filter may be a mechanical filter.

The mechanical filter may be a HEPA filter. The dust bin may be removable from the housing.

The dust bin may have a lid to enable emptying and cleaning of the bin, and replacement of the filters.

The housing may have a bottom side parallel to and spaced above the surface, and the intake opening may be disposed therethrough.

The appliance may have rotating brushes depending from the bottom side and extending outwardly from the housing between the bottom side and the surface and adapted to force germs and germ-laden dust from beyond the housing and in surface corners to under the housing and towards the intake opening. The appliance may have a rotating roller brush in communication with the intake opening and adapted to feed germs and germ-laden dust there-into.

The housing may have one or more perimetral sides and a top side and the exhaust opening may be disposed through one or both thereof. The housing may include a battery for proving power to the appliance. The battery may be a rechargeable battery.

The appliance may include a stationary recharging station separable from the housing and adapted to receive the housing and recharge the battery. The housing may include a computer including a first Wifi transmitter/receiver and first portion of an operating program.

The appliance may have a remote controller including a second Wifi transmitter/receiver and a second portion of the operating system. The remote controller may be a smartphone, and the second portion of the operating system may be a smartphone app downloadable from the Internet.

The dust bin may be made of a UV-resistant polymer, adapted to seal UV light therein, and adapted so that all of the germs and germ-laden dust which enter the bin is exposed thereto.

The appliance may have a motorized transport system adapted to move the appliance about the surface.

The appliance may have an ion generator and an essential oil diffuser in communication with the exhaust opening; wherein the air leaving the bin through the exhaust opening passes the ion generator and the essential oil infuser and is ionized and infused as it exists the housing.

The HEPA filter may be at least HEPA-13 grade or equivalent.

Further features and aspects of the invention are disclosed with more specificity in the Description and Drawings provided herein and showing exemplary embodiments of the invention.

### BRIEF DESCRIPTION OF THE DRAWINGS

An exemplary embodiment of a germicidal floor cleaning appliance in accordance with or useful in practicing the invention is shown in the accompanying Drawings, of which;

FIG. 1 is a perspective view of an exemplary floor cleaning appliance;

FIG. 2 is a top view of the appliance of FIG. 1;

FIG. 3 is a front view of the appliance of FIG. 1;

FIG. 4 is a side view of the appliance of FIG. 1;

FIG. 5 is a bottom view of the appliance of FIG. 1;

FIG. 6 is an exploded view of some of the key components of the appliance of FIG. 1;

FIG. 7 is a perspective cross-sectional view of the appliance of FIG. 1 taken at Line 7-7 of FIG. 2;

FIG. 8 is a perspective cross-sectional view of the appliance of FIG. 1 taken at Line 8-8 of FIG. 2;

FIG. 9 is a perspective cross-sectional view of the appliance of FIG. 1 taken at Line 9-9 of FIG. 2;

FIG. 10 is an exploded view of the water tank of the appliance of FIG. 1;

FIG. 11 is a perspective view of the assembled water tank of FIG. 10 in its open state;

FIG. 12A is a perspective view of the appliance of FIG. 1 approaching its docking station;

FIG. 12B is a front perspective view of the appliance of FIG. 1;

FIG. 13 is a first smartphone screen image during installation of the application program for controlling the appliance of FIG. 1;

FIG. 14 is a second smartphone screen image during installation of the application program for controlling the appliance of FIG. 1;

FIG. 15 is a third smartphone screen image during installation of the application program for controlling the appliance of FIG. 1;

FIG. 16 is a fourth smartphone screen image during installation of the application program for controlling the appliance of FIG. 1;

FIG. 17 is a fifth smartphone screen image during installation of the application program for controlling the appliance of FIG. 1;

FIG. 18 is a sixth smartphone screen image during installation of the application program for controlling the appliance of FIG. 1;

FIG. 19 is a seventh smartphone screen image during installation of the application program for controlling the appliance of FIG. 1;

FIG. 20 is an eighth smartphone screen image during installation of the application program for controlling the appliance of FIG. 1;

FIG. 21 is a ninth smartphone screen image during installation of the application program for controlling the appliance of FIG. 1;

FIG. 22 is a tenth smartphone screen image during installation of the application program for controlling the appliance of FIG. 1;

FIG. 23 is an eleventh smartphone screen image during installation of the application program for controlling the appliance of FIG. 1;

FIG. 24 is a twelfth smartphone screen image during installation of the application program for controlling the appliance of FIG. 1;

FIG. 25 is a first smartphone screen image during operation of the application program for controlling the appliance of FIG. 1;

FIG. 26 is a second smartphone screen image during operation of the application program for controlling the appliance of FIG. 1;

FIG. 27 is a third smartphone screen image during operation of the application program for controlling the appliance of FIG. 1;

FIG. 28 is a fourth smartphone screen image during operation of the application program for controlling the appliance of FIG. 1;

FIG. 29 is a fifth smartphone screen image during operation of the application program for controlling the appliance of FIG. 1;

FIG. 30 is a sixth smartphone screen image during operation of the application program for controlling the appliance of FIG. 1;

FIG. 31 is a seventh smartphone screen image during operation of the application program for controlling the appliance of FIG. 1;

FIG. 32 is an eighth smartphone screen image during operation of the application program for controlling the appliance of FIG. 1;

FIG. 33 is a ninth smartphone screen image during operation of the application program for controlling the appliance of FIG. 1;

FIG. 34 is a tenth smartphone screen image during operation of the application program for controlling the appliance of FIG. 1;

FIG. 35 is an eleventh smartphone screen image during operation of the application program for controlling the appliance of FIG. 1;

FIG. 36 is a twelfth smartphone screen image during operation of the application program for controlling the appliance of FIG. 1;

FIG. 37 is a thirteenth smartphone screen image during operation of the application program for controlling the appliance of FIG. 1;

FIG. 38 is a fourteenth smartphone screen image during operation of the application program for controlling the appliance of FIG. 1; and

FIG. 39 is a fifteenth smartphone screen image during operation of the application program for controlling the appliance of FIG. 1.

### DESCRIPTION OF AN EXEMPLARY EMBODIMENT

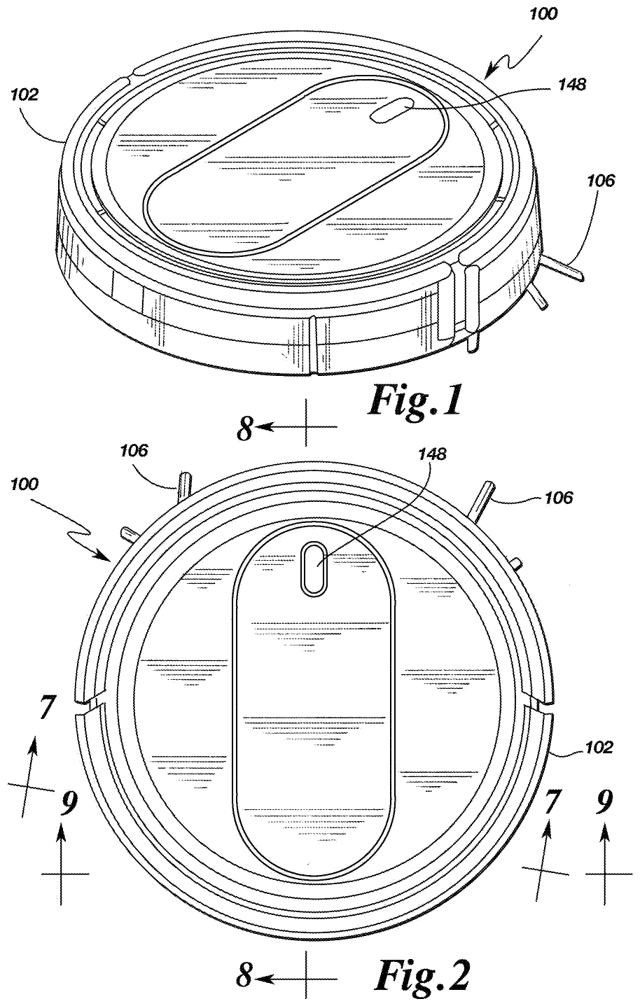

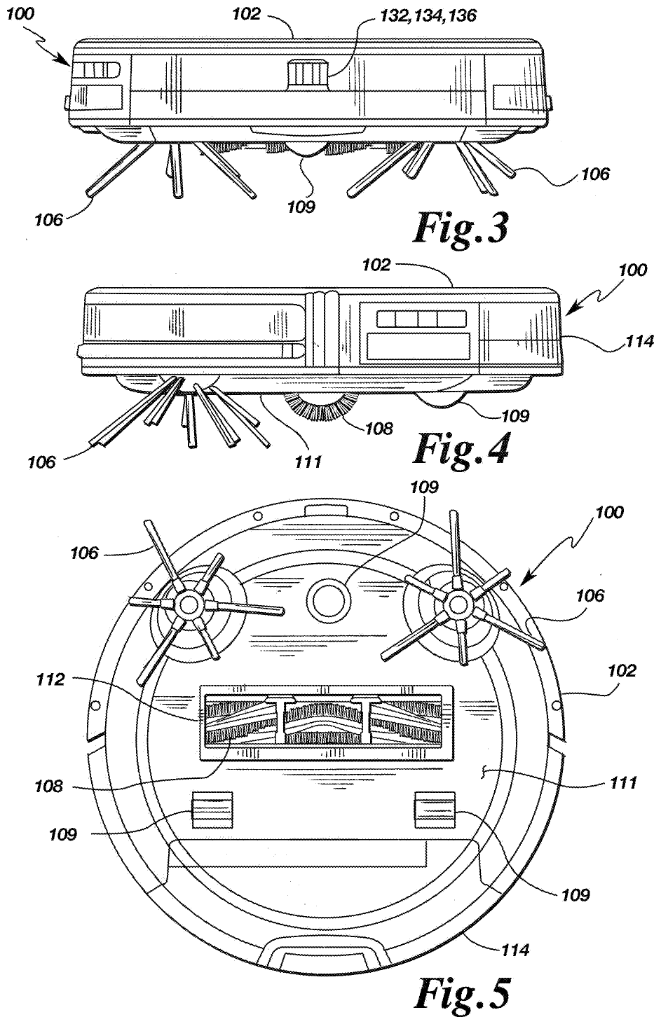

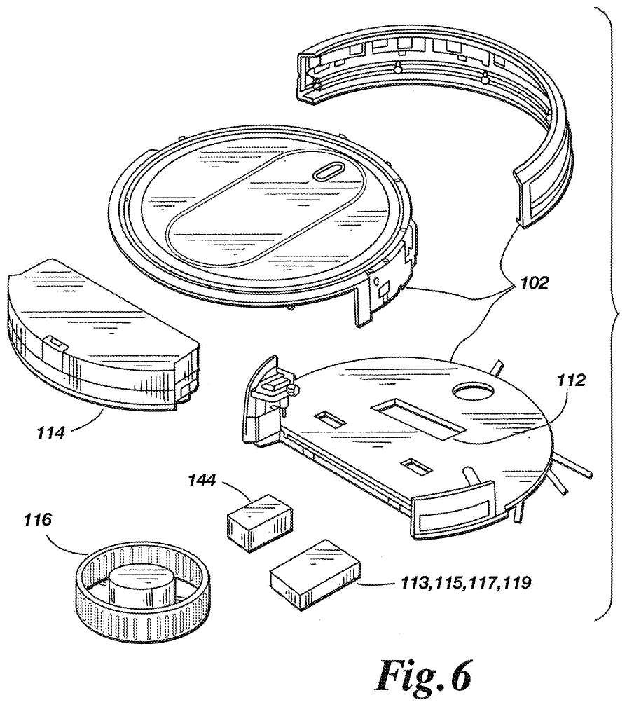

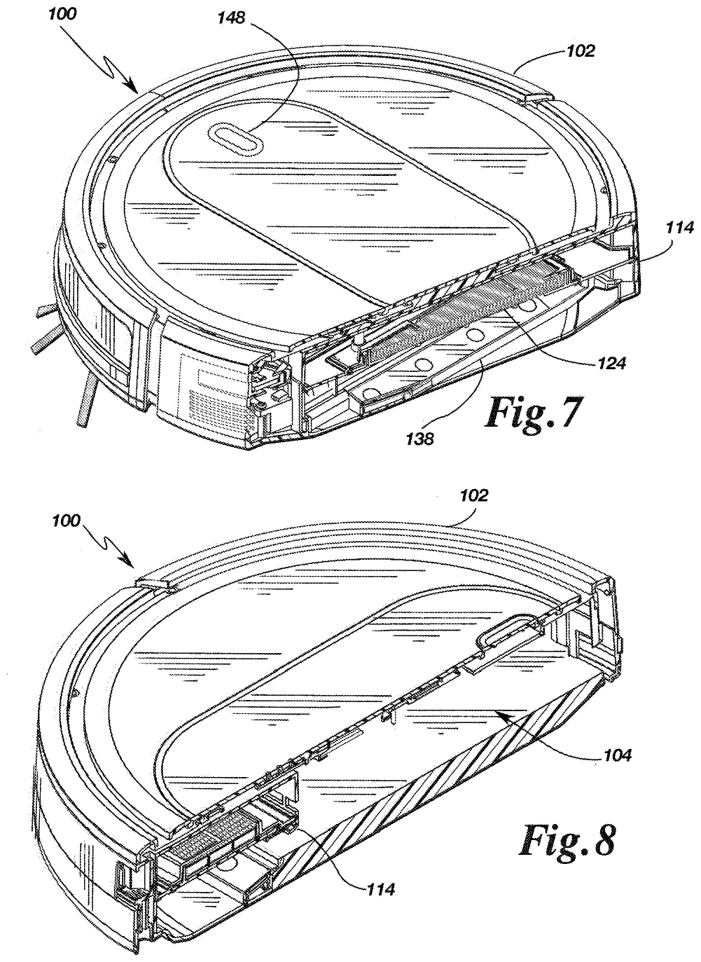

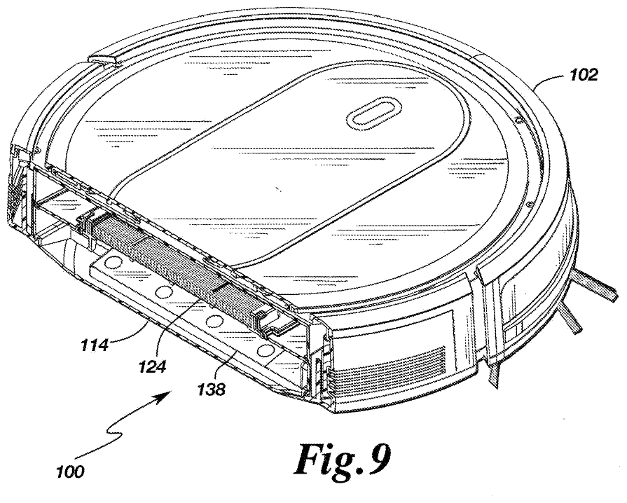

Referring to FIGS. 1 through 12B there is shown a floor cleaning appliance in the form of a robotic vacuuming floor sweeping system including sweeper 100 and a recharging station 200. The sweeper's housing 102 defines a chamber 104. The housing has vertically-rotating brushes 106 depending from its bottom side which are adapted to force germs and germ-laden dust from beyond the housing and in floor-to-wall corners to under the housing. The housing has a horizontally-rotating roller brush 108 adapted to force that debris and any germs and germ-laden dust it scrubs from the floor under the housing and feeds it into intake opening 112.

The housing is moved about the floor by means of a transportation system including an internal motor which drives wheels 109 which protrude from the housing's underside 111. The housing includes an internal control module that includes computer 113, Wifi transmitter/receiver 115, Wifi hotspot 117, and an appliance portion of an operating program 119.

The housing includes a dust bin 114 adapted for collecting and removing the germs and germ-laden dust taken from the floor. Motorized blower 116 pulls air including the germs and germ-laden dust in through the intake opening, and into the dust bin. The bin includes a chamber 118 and lid 128 that is equipped with a series of UVC lamps 138. As the air is circulated within the chamber, the UV is able to disinfect it over an extended period of time for optimal efficiency. Then the air passes through and the germs and germ-laden dust come in contact with the bactericidal copper mesh filter 122 where most germs are killed, and then the air passes through the ultrafine HEPA-13 filter 124 where smaller where smaller particles get retained. The air then leaves the bin as virtually germ-free and particle-free air and passes the ion generator 132 and essential oil infuser 134 and then out of the housing through exhaust opening 136.

The incoming air is first treated by exposure of its germs to UV-C radiation from the lamps. This kills virtually all germs. The shape and size of the chamber in relation to the intake and exhaust openings results in the air being circulated around and around therein for a prolonged amount of time to increase the UV exposure time and maximize germ killing. The UV light also bathes any dust within the air. The dust bin is made of a polymer which is treated to resist the decomposing effects of UV light and seals the UV radiation within the bin.

Live germs which contact the copper mesh filter are killed by contact. Copper ions emitted by the filter are molecularly attracted to other germs passing through the filter and are killed by a combination of cell wall penetration and DNA alteration. The ions also charge the germs and dust to increase their retention by the HEPA filter, which the air is next passed through.

The medical-grade HEPA-13 filter collects all particles greater than 0.1 microns in diameter. This includes all dust and most dead germs and germ parts. Dust collects on the HEPA filter and falls to the floor of the bin. Most particles smaller than 0.1 microns which pass through the HEPA filter are killed by the UV light exposure and contact with the copper filter.

Air leaving the dust bin may still contain a very low amount of very tiny inorganic particulate matter which even the finest filtration could not remove, and which is unaffected by UV radiation. These particulates are treated with an essential oil as they leave the bin, which eliminates their odor and provides them with freshness. These particulates are then exposed to ion generation before being exhausted from the housing. The ions are attracted to and molecularly-bond with the particulates, causing them to take a charge and thus become attracted to nearby surfaces and objects, where their fresh odor improves the atmosphere in the room. The essential oil diffuser will disperse the essential oil whenever the appliance is running, until the oil is depleted.

The housing also includes a rechargeable Li-Ion battery 144, battery terminals, and control panel 148 including “Home” switch 154, “Power” switch 115, and an indicator light.

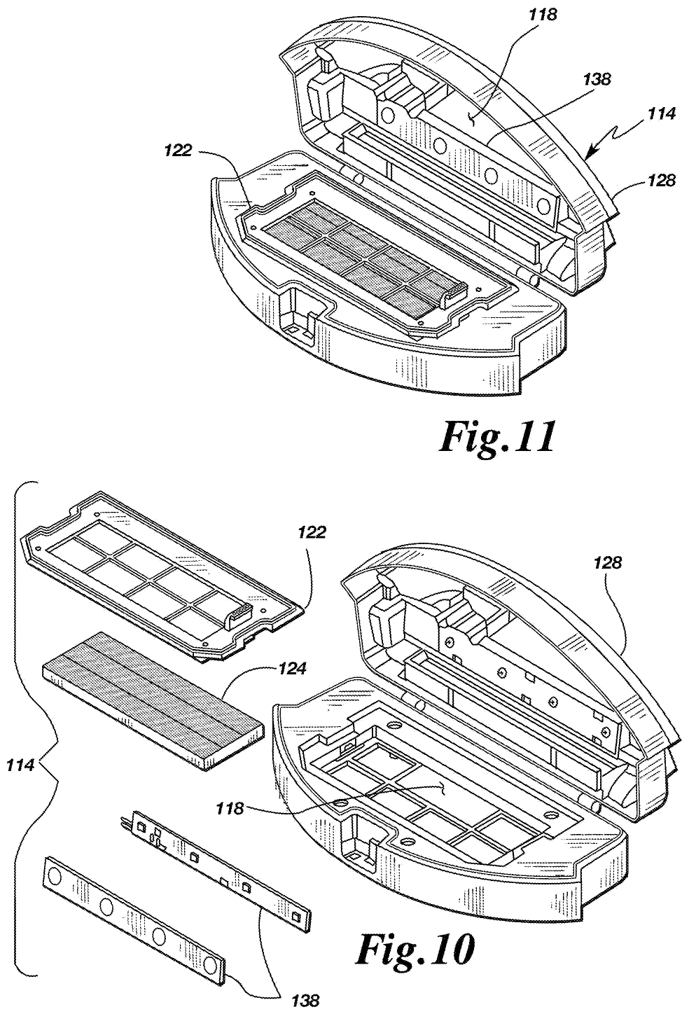

FIGS. 10 and 11 show the removable dust bin including the UV chamber, UV-C lamps, Copper filter, HEPA-13 filter, and lid. The bin is removed from the housing for emptying and for changing the filters as needed.

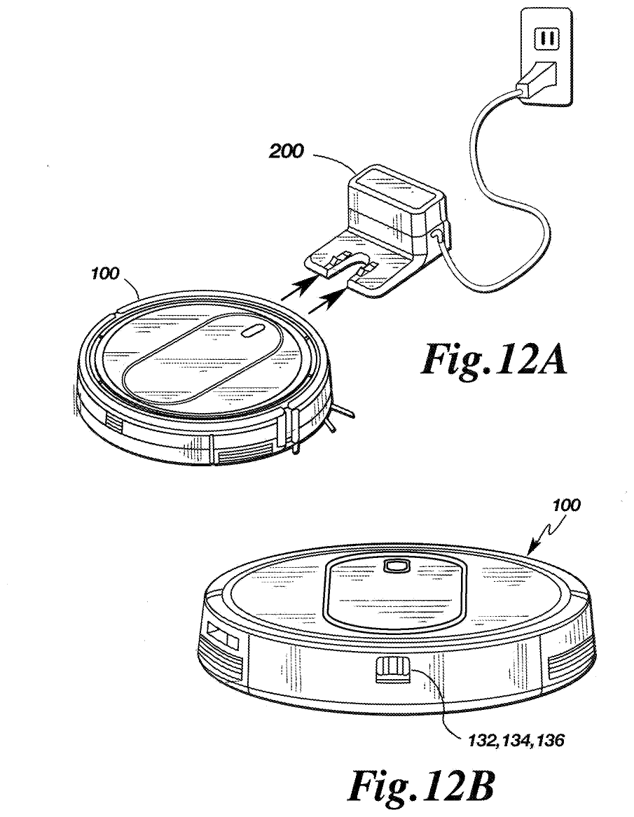

FIG. 12A depicts the sweeper approaching charging station (or “home base”) 200. The sweeper “knows” the location of the station by a signal sent therefrom, and the sweeper returns to the station at the earliest of completion of the assigned cleaning task, when the dust bin or one or its filters are sensed full, or when its battery requires recharge. The station has electrical terminal pads which are contacted by mating terminal pads on the underside of the sweeper.

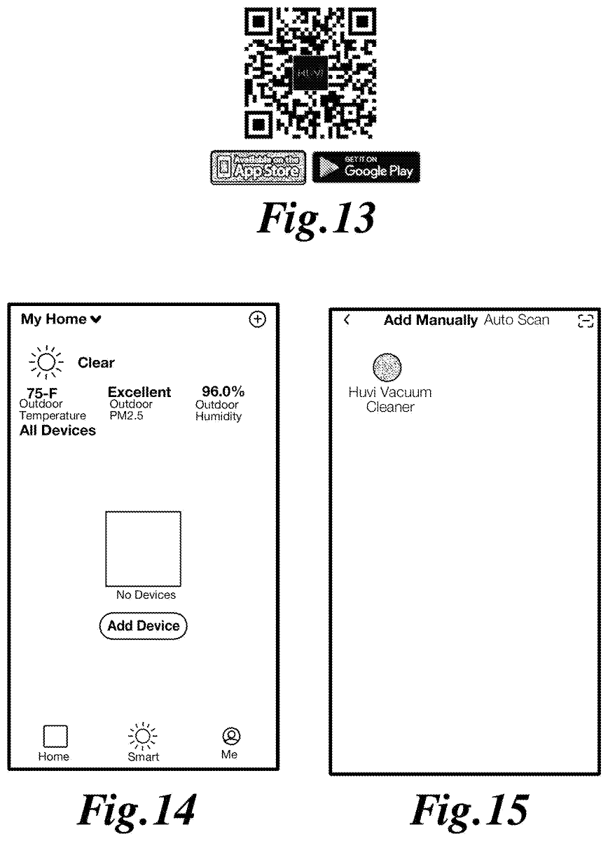

Before using the appliance, an app can be downloaded and installed to the user's smart phone. Documentation supplied with the appliance provides a QR Code (FIG. 13) which is scanned to bring the user to the app for doing this.

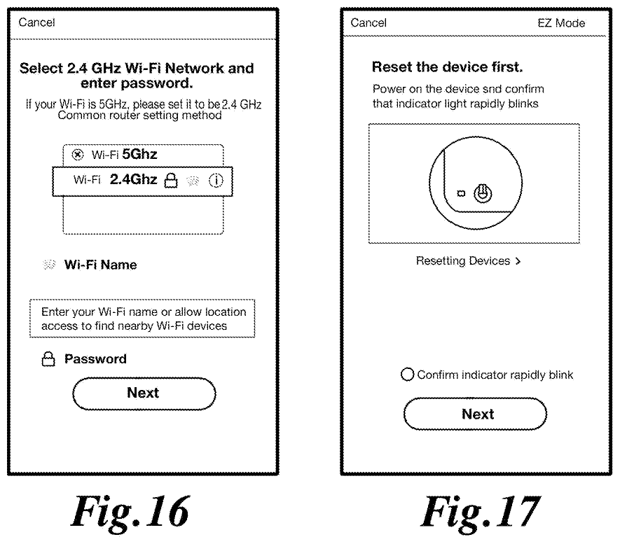

Once the download is complete, the app is opened, and the user is prompted to create a personal account. The user is then prompted to add the appliance to the app (FIGS. 14 and 15) and to connect the device to the prevailing Wifi network (FIG. 16). The user is prompted to choose between an “EZ Mode” or an “AP Mode” (FIG. 17).

The EZ Mode uses the home's WiFi network to communicate between the smartphone and the appliance, while the AP Mode uses the appliance's built-in WiFi hotspot to communicate between the smartphone and the appliance.

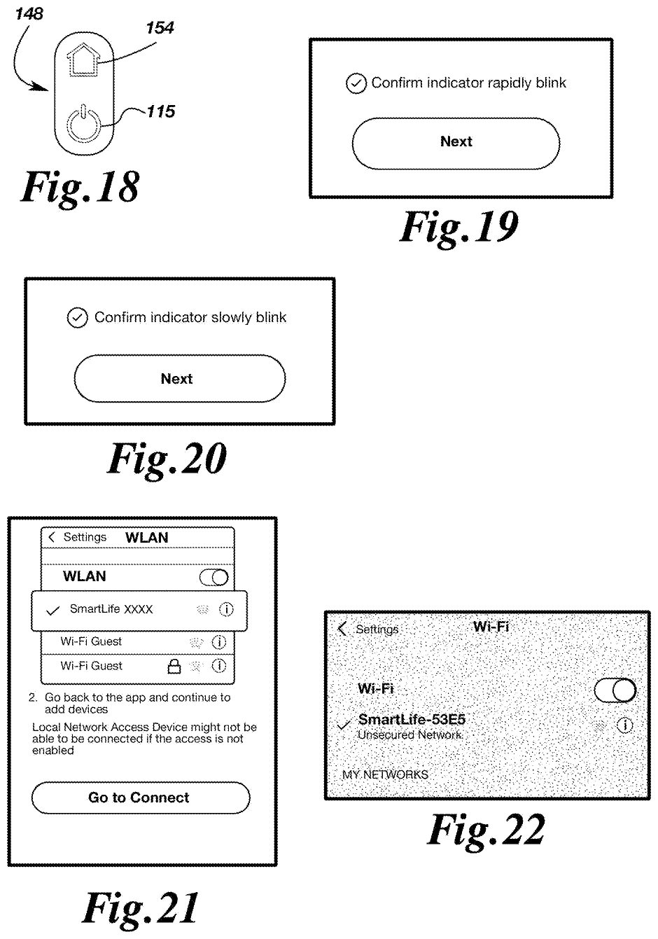

For connecting in EZ Mode, the user presses and holds the “Home” button on the appliance indicated by a house-shaped icon (FIG. 18) after selecting “EZ Mode”, which causes connection to the home's Wifi network and causes the appliance's voice transmitter to say “Connecting to Network” and the app to indicate “Adding Device”. Once complete, the Home button glows light blue during this connection and flashes rapidly. The user is meanwhile prompted to confirm that the indicator light on the appliance is rapidly flashing (FIG. 19). Once connected, the user is prompted to assign a nickname to the appliance and then select “Done” in the app to complete. The appliance is now controlled through the app.

For connecting in AP Mode, such as if connection in EZ mode connection was unsuccessful, the user presses and holds the Home button after selecting “AP Mode”, which causes connection to the appliance's Wifi hotspot, and causes the appliance's voice transmitter to say “Connecting to Network” and the app to indicate “Adding Device”. Once complete, the Home button glows light blue during this connection and flashes slowly. The user is meanwhile prompted to confirm that the indicator light on the appliance is slowly flashing (FIG. 20). The WiFi's hotspot appears, and the user selects it and turns it on (FIGS. 21 and 22). Once connected, the user is prompted to assign a nickname to the appliance and then select “Done” in the app to complete. The appliance is now controlled through the app.

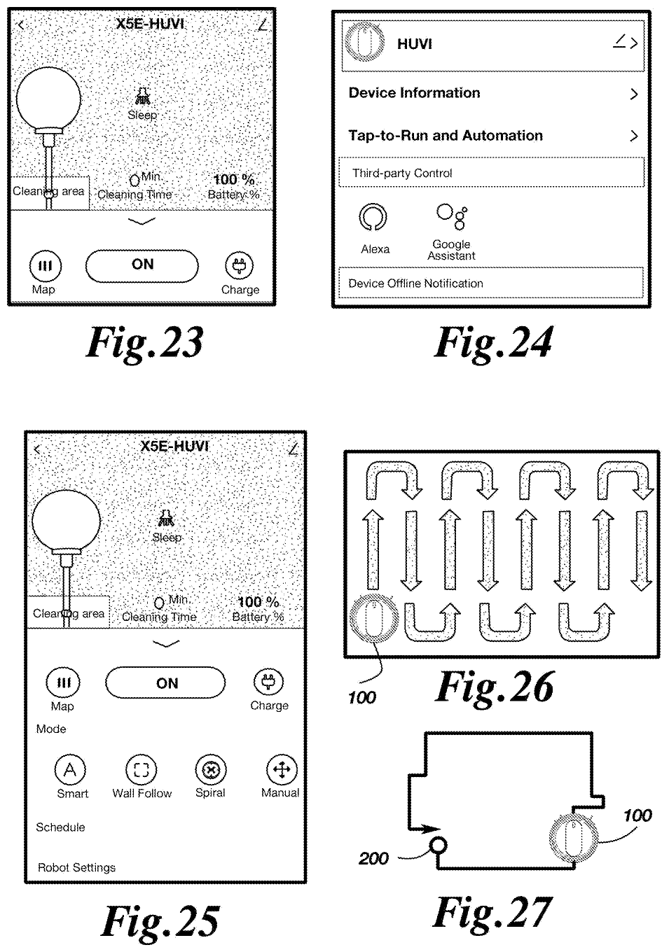

The appliance is compatible with Amazon Echo's Alexa. With the appliance turned on (FIG. 23) the “Alexa” icon is selected in the app's Control screen (FIG. 24) and instructions are provided to connect. The appliance is also compatible with Google Home by instead similarly selecting the “Google Assistant” icon in the app's control screen.

The appliance can be directed to clean and recharge by using the POWER and HOME buttons (FIG. 18). Tapping the Home button once will send the appliance to the charging station 200 where it's battery will be charged to full capacity. Holding the Home button for 3-5 seconds will cause toggling between the EZ and AP modes of operation. Holding the Power button for 3-5 seconds will power the appliance on/off. Tapping the Power Button quickly will scroll the appliance through its smart cleaning function; Start/Pause/Resume.

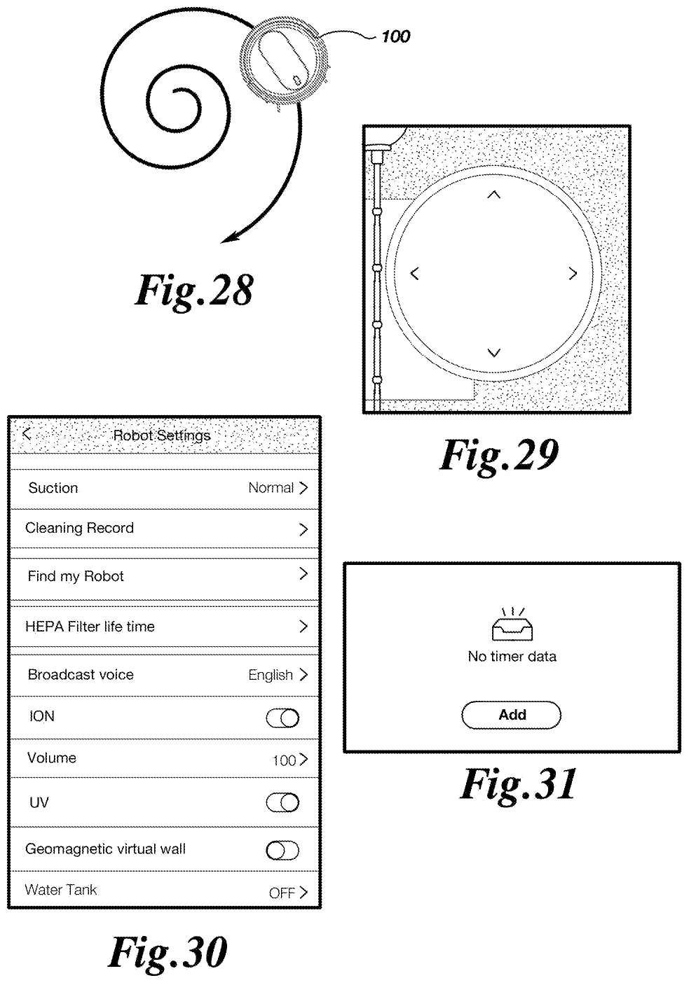

The app provides a full array of functions and controls. Referring to the app's Home screen (FIG. 25), selection of the “ON” button after selecting the desired function will cause the cleaning according to user preference. In Smart mode, the appliance navigates and clean the floor following the path shown in FIG. 25. In Wall Follow mode, the appliance reduces its speed when it detects a wall and then follows the wall to ensure the edge is cleaned thoroughly (FIG. 27). In Spot mode, the appliance moves in an outward spiral, cleaning the floor in expanding circles (FIG. 28). And in Manual Control mode the user is enables to “drive” the appliance in desired directions (FIG. 29). The appliance will automatically return to the charging base when it senses a low battery while running.

The appliance has 4 suction power settings: Low, Medium, High, and Max. Choosing “Robot Settings” in the Home screen allows selection between those (FIG. 30). The suction setting can be changed during any of the other operating functions.

In “Robot Settings” the user can select “UV” to toggle the UV light on or off. This function is enabled by default when first powering on the appliance. The user can select “ION” to toggle the Ion Generator on or off. These functions are enabled by default when first powering on the appliance.

The user may schedule cleaning by selecting “Schedule” in the Home screen, then selecting “Add” (FIG. 31) where he will be prompted to select a starting time and other parameters (FIG. 32) and then saving.

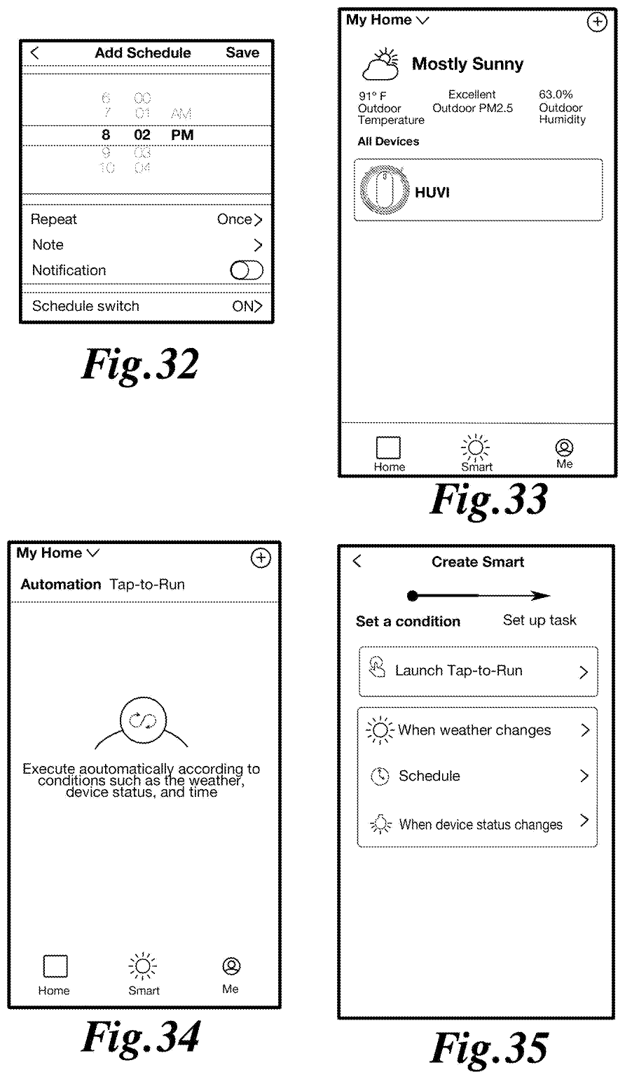

The appliance may be set to run automatically according to parameters selected. For instance, the appliance can be set to operate according to certain weather conditions, appliance status, or time of day. Selecting the “Smart” icon (FIG. 33) opens the screen of FIG. 34 where the automated “Tap-to-Run” function is activated. Launching the function allows the user to set commands which the appliance will follow with one touch on the app (FIG. 35). For example, “When weather changes” allows control of operation according to the weather. “Schedule” allows setting of a schedule for the performance of specific tasks. “When the device status changes” allows the user to set commands depending on the appliance's status. For example, the appliance can be directed to return to the charging station according to the remaining filter life. After setting a condition, a task is selected (FIG. 36) and onscreen instructions are then followed.

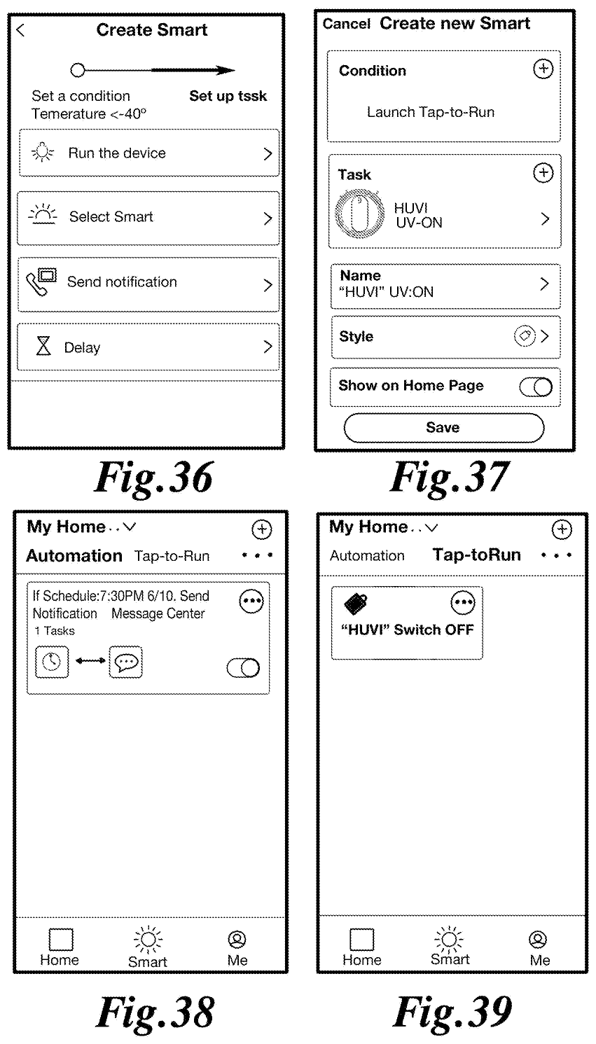

“Run the device” allows the user to select which functions to enable when automating. “Select Smart” allows the user to select an Automation or Tap-to-Launch that had previously been created. “Send notification” will send a notification to the smartphone when the set condition is met. “Delay” allows a delay to the start of the automation by a specific amount of time. After setting a task, further conditions/tasks may be added if desired. When ready, “Save” is selected to finalize (FIG. 37).

Once created, “Automations” can be found by selecting “Automations” in the Smart Screen (FIG. 38). “Tap-to-Run” commands can be found by selecting “Tap-to Run” section in the Smart Screen (FIG. 39).

Key Aspects of the appliance include;
4″ by 6″ vertical-axis rotating brushes reach into corners to loosen germs stuck there and pull in them towards the appliance intake,
Dual stiffness horizontal-axis roller brush directs germs and particles into the intake,
Vacuum pulls the germs and particles from floor and into the appliance,
Copper filter kills up to 99% of bacteria and viruses in incoming air on contact. Copper ions destroy the outer membrane of the pathogen and damage its DNA,
HEPA-13 Medical Grade filter removes 99.95% of germs down to 0.1 microns in diameter,
Bipolar Ionization kills germs by emitting opposing magnetic charges which create polarized ions that are attracted to germs and destroy their cell walls and chemical bonds within pathogens . . . releasing only water molecules as a byproduct,
Removable 600 mL dust bin stores particles not trapped by filtration,
UV-C light kills mold, bacteria, VOCs, odors, and most allergens in the airflow and residing within the appliance. Germs caught by the HEPA filter are continuously bathed in UV-C and killed. Dust collected by the appliance is continuously bathed in UV-C and sterilized to avoid exposure when emptying,
Essential Oil diffuser adds healthful freshness to the exhausting air,
Smartphone application is downloadable from Google Play Store for Android phones and from Apple App Store for I-phones and communicates through the phones by both Bluetooth and wireless cellular signal or through Alexa and Google Home,
3200 mAh Lithium battery.

While the invention has been shown and described with reference to a specific exemplary embodiment, it should be understood by those skilled in the art that various changes in form and detail may be made without departing from the spirit and scope of the invention. For instance, the germicidal system may equally be used in a robotic vacuum, a long-handles floor sweeper, an upright or canister vacuum, or any similar floor cleaning appliance. The invention should therefore only be limited according to the following claims, including all equivalent interpretation to which they are entitled.

1. A surface cleaning appliance comprising:
a housing comprising;
an intake opening;
an exhaust opening;
a dust bin in communication with and disposed between the intake and exhaust openings and adapted for retaining airborne germs and germ-laden dust; and
a motorized blower adapted to pull air containing the germs and germ-laden dust into the dust bin through the intake opening where the germs and germ-laden dust are removed therefrom, and to expel the air from the dust bin through the exhaust opening; wherein
the dust bin comprises a UV lamp and first and second filters; and
the air containing the germs and germ-laden dust is pulled into the dust bin and through the intake opening where it is bathed in UV light from the UV lamp, passes through the first and second filters, and leaves the bin through the exhaust opening.

2. The surface cleaning appliance of claim 1 where the dust bin comprises a chamber adapted so that air is circulated around therein for a prolonged period to increase direct UV exposure thereof.

3. The surface cleaning appliance of claim 2 wherein the UV lamp also directly bathes interior portions and components of the bin to expose any germs retained therein to the UV light.

4. The surface cleaning appliance of claim 3 wherein the UV light is UV-C.

5. The surface cleaning appliance of claim 4 wherein the first filter is a germicidal filter.

6. The surface cleaning appliance of claim 5 wherein the germicidal filter is a copper mesh filter.

7. The surface cleaning appliance of claim 6 wherein the second filter is a mechanical filter.

8. The surface cleaning appliance of claim 7 wherein the mechanical filter is a HEPA filter.

9. The surface cleaning appliance of claim 8 wherein the dust bin is removable from the housing.

10. The surface cleaning appliance of claim 9 wherein the dust bin further comprises a lid to enable emptying and cleaning of the bin, and replacement of the filters.

11. The surface cleaning appliance of claim 10 wherein the housing further comprises a bottom side parallel to and spaced above the surface, and the intake opening is disposed there-through.

12. The surface cleaning appliance of claim 11 further comprising rotating brushes depending from the bottom side and extending outwardly from the housing between the bottom side and the surface and adapted to force germs and germ-laden dust from beyond the housing and in surface corners to under the housing and towards the intake opening.

13. The surface cleaning appliance of claim 12 further comprising a rotating roller brush in communication with the intake opening and adapted to feed germs and germ-laden dust there-into.

14. The surface cleaning appliance of claim 13 wherein the housing further comprises one or more perimetral sides and a top side, and the exhaust opening is disposed through one or both thereof.

15. The surface cleaning appliance of claim 14 wherein the housing further comprises a battery for proving power to the appliance.

16. The surface cleaning appliance of claim 15 wherein the battery is a rechargeable battery.

17. The surface cleaning appliance of claim 16 wherein the appliance further comprises a stationary recharging station separable from the housing and adapted to receive the housing and recharge the battery.

18. The surface cleaning appliance of claim 17 wherein the housing further comprises a computer including a first Wifi transmitter/receiver and a first portion of an operating program.

19. The surface cleaning appliance of claim 18 wherein the appliance further comprises a remote controller comprising a second Wifi transmitter/receiver and a second portion of the operating system.

20. The surface cleaning appliance of claim 19 wherein the remote controller is a smartphone, and the second portion of the operating system is a smartphone app downloadable from the Internet.

21. The surface cleaning appliance of claim 20 wherein the dust bin is comprised of a UV-resistant polymer, is adapted to seal UV light therein, and is adapted so that all of the germs and germ-laden dust which enter the bin is exposed thereto.

22. The surface cleaning appliance of claim 21 further comprising a motorized transport system adapted to move the appliance about the surface.

23. The surface cleaning appliance of claim 22 further comprising:
an ion generator and an essential oil diffuser in communication with the exhaust opening; wherein
the air leaving the bin through the exhaust opening passes the ion generator and the essential oil infuser and is ionized and infused as it exists the housing.

24. The surface cleaning appliance of claim 23 wherein the HEPA filter is at least HEPA-13 grade or equivalent.
